(19) United States
(12) Patent Application Publication
(10) Pub. No.: US 2022/0047260 A1
(43) Pub. Date: Feb. 17, 2022

(54) CHIPPED TROCAR ASSEMBLY FOR CIRCULAR STAPLING INSTRUMENTS

(71) Applicant: Covidien LP, Mansfield, MA (US)

(72) Inventor: Joseph EISINGER, Northford, CT (US)

(21) Appl. No.: 17/400,534

(22) Filed: Aug. 12, 2021

Related U.S. Application Data
(60) Provisional application No. 63/064,572, filed on Aug. 12, 2020.

(51) Int. Cl. A61B 17/068; A61B 17/34; A61B 90/00

(52) U.S. Cl. CPC A61B 17/068; A61B 17/3476; A61B 2017/00221; A61B 90/08; A61B 17/3478

(57) ABSTRACT

An adapter assembly includes a trocar assembly releasably securable within an elongate body and including a chip. The adapter assembly may further include a housing supporting a circuit board. The chip electrically communicates with the circuit board when the trocar assembly is received within the distal portion of the elongate body.

### CROSS-REFERENCE TO RELATED APPLICATION

This application claims the benefit of and priority to U.S. Provisional Patent Application No. 63/064,572, filed Aug. 12, 2020, the entire contents of which is incorporated by reference herein.

### FIELD

The disclosure relates to reusable adapter assemblies for surgical stapling devices. More particularly, the disclosure relates to a trocar assembly having a chip for use with a reusable adapter assembly.

### BACKGROUND

Surgical devices for applying staples, clips, or other fasteners to tissue are well known. Typically, endoscopic stapling devices include an actuation unit, e.g., a handle assembly for actuating the device and a shaft for endoscopic access, and a tool assembly disposed at a distal end of the shaft. In certain of these devices, the shaft includes an adapter assembly, having a proximal end securable to the handle assembly and a distal end securable to the tool assembly.

Circular stapling devices typically include a trocar assembly for supporting an attached anvil assembly. The trocar assembly may be releasably securable within the adapter assembly to permit cleaning, sterilization, and reuse of the adapter assembly.

### SUMMARY

An adapter assembly for connecting a loading unit to a handle assembly includes an elongate body having a proximal portion and a distal portion. The proximal portion is configured for operable engagement with an actuation assembly and the distal portion defines a longitudinal axis. The adapter assembly further includes a trocar assembly releasably receivable within the distal portion of the elongate body. The trocar assembly includes a chip for storing data.

In certain aspects of the disclosure, the adapter assembly further includes a housing supporting a circuit board. The chip electrically communicates with the circuit board when the trocar assembly is received within the distal portion of the elongate body. The housing may be a component of a strain gauge assembly. The trocar assembly may include a trocar housing, a trocar member, and a drive screw. Rotation of the drive screw in a first direction causes advancement of the trocar member relative to the trocar housing and rotation of the drive screw in a second direction causes retraction of the trocar member relative to the trocar housing.

In some aspects of the disclosure, the data includes a length of the trocar member. The drive screw may include a thread having a pitch. The data includes the pitch of the drive screw. The data may include a diameter of the trocar member. The data may include a number of times the trocar assembly has been used. The trocar assembly may include first and second contact members and the elongate body may include first and second contact members. The first and second contact members of the trocar assembly may be configured to engage with the respective first and second contact members of the elongate body when the trocar assembly is received within the distal portion of the elongate body.

In other aspects of the disclosure, the adapter assembly further includes a circuit board assembly in electric communication with the chip when the trocar assembly is received within the distal portion of the adapter assembly.

A surgical stapling instrument includes an actuation assembly and an adapter assembly for connecting a loading unit to the actuation assembly. The adapter assembly includes an elongate body having a proximal portion and a distal portion. The proximal portion is configured for operable engagement with an actuation assembly and the distal portion defines a longitudinal axis. The adapter assembly further includes a trocar assembly releasably receivable within the distal portion of the elongate body. The trocar assembly includes a chip for storing data.

In certain aspects of the disclosure, the adapter assembly further includes a housing supporting a circuit board. The chip electrically communicates with the circuit board when the trocar assembly is received within the distal portion of the elongate body. The housing may be a component of a strain gauge assembly. The trocar assembly may include a trocar housing, a trocar member, and a drive screw. Rotation of the drive screw in a first direction may cause advancement of the trocar member relative to the trocar housing and rotation of the drive screw in a second direction may cause retraction of the trocar member relative to the trocar housing.

In some aspects of the disclosure, the data includes a length of the trocar member. The drive screw may include a thread having a pitch. The data includes the pitch of the drive screw. The data may include a diameter of the trocar member. The data may include a number of times the trocar assembly has been used. The trocar assembly may include first and second contact members and the elongate body includes first and second contact members, the first and second contact members of the trocar assembly being configured to engage with the respective first and second contact members of the elongate body when the trocar assembly is received within the distal portion of the elongate body.

In other aspects of the disclosure, the adapter assembly further includes a circuit board assembly in electric communication with the chip when the trocar assembly is received within the distal portion of the adapter assembly.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiments given below, serve to explain the principles of the disclosure, wherein:

FIG. 1 is a perspective view of a surgical stapling device including a handle assembly and an adapter assembly according to an exemplary embodiment of the disclosure;

FIG. 2 is a perspective view of the adapter assembly shown in FIG. 1 with a trocar assembly extending from a distal portion of the adapter assembly;

FIG. 3 is a perspective view of the distal end of the adapter assembly and the trocar assembly shown in FIG. 2, with the trocar assembly separated from the adapter assembly;

FIG. 4 is an enlarged view of the indicated area of detail shown in FIG. 3;

FIG. 5 is a side perspective view of a housing of a strain gauge assembly of the adapter assembly shown in FIG. 2;

FIG. 6 is a cross-sectional end view of the adapter assembly shown in FIG. 3, taken along section line 6-6 shown in FIG. 3;

FIG. 7 is a cross-sectional side view of the adapter assembly shown in FIG. 3, taken along section line 7-7 shown in FIG. 6;

FIG. 8 is a cross-sectional end view of the adapter assembly shown in FIG. 2, taken along section line 8-8 shown in FIG. 2; and

FIG. 9 is a cross-sectional side view of the adapter assembly shown in FIG. 2, taken along section line 9-9 shown in FIG. 2.

### DETAILED DESCRIPTION

Embodiments of the disclosed adapter assembly including a retaining mechanism for securing a removable trocar assembly therein will now be described in detail with reference to the drawings in which like reference numerals designate identical or corresponding elements in each of the several views. As is common in the art, the term “proximal” refers to that part or component closer to the user or operator, i.e. surgeon or clinician, while the term “distal” refers to that part or component further away from the user.

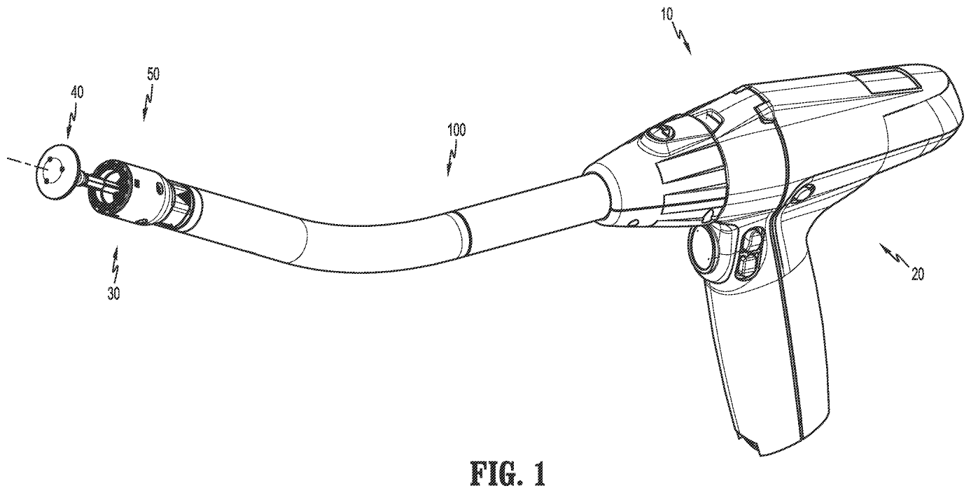

FIG. 1 illustrates a surgical stapling device 10 including an adapter assembly according to an exemplary embodiment of the disclosure, shown generally as adapter assembly 100. The surgical stapling device 10 further includes a powered handle assembly 20, a loading unit 30, and an anvil assembly 40. The loading unit 30 and anvil assembly 40 together form an end effector 50. Although shown and described with reference to surgical stapling device 10, aspects of the disclosure may be modified for use with manual surgical stapling devices having various configurations, and with powered surgical stapling devices having alternative configurations. For a detailed description of exemplary surgical stapling devices, please refer to U.S. Pat. No. 9,023,014 and U.S. Pat. Appl. Publ. No. 2012/0253329. It is also envisioned that the aspects of the disclosure may be modified for use in robotic systems, or other remotely controlled operating systems.

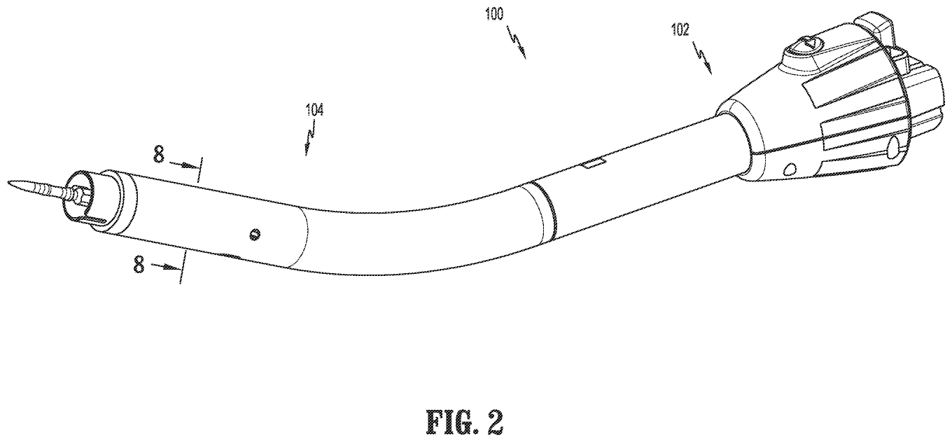

FIG. 2 illustrates the adapter assembly 100 including an elongate body having a proximal portion 102 configured for operable connection to the handle assembly 20 (FIG. 1) and a distal portion 104 configured for operable connection to the loading unit 30 (FIG. 1) and to the anvil assembly 40 (FIG. 1). Although shown and described as forming an integral unit, it is envisioned that the proximal and distal portions 102, 104 may be formed as separate units that are releasably securable to one another.

The adapter assembly 100 will only be described to the extent necessary to fully disclose the aspects of the disclosure. For a detailed description of an exemplary adapter assembly, please refer to U.S. Pat. No. 10,226,254 (“the '254 patent).

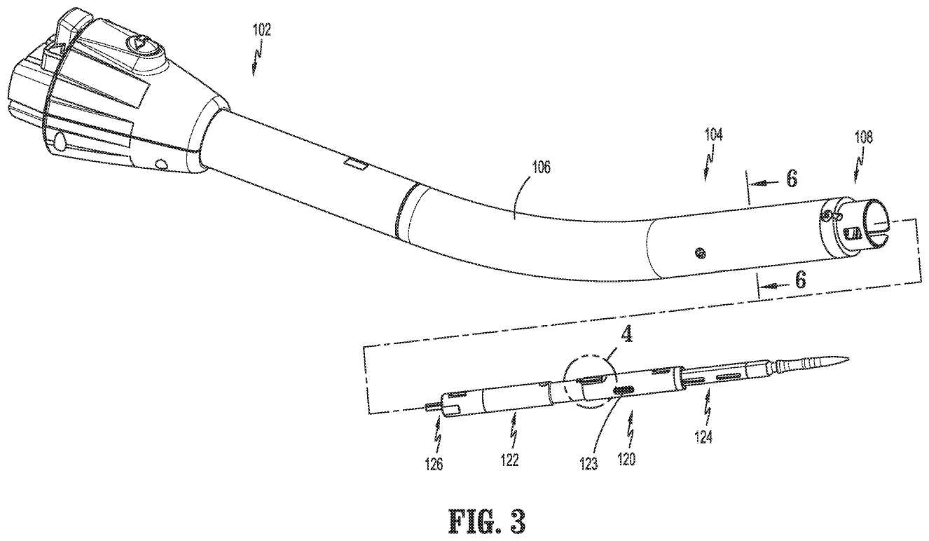

FIG. 3 illustrates the adapter assembly 100 including an outer sleeve 106, and a connector housing 108 secured to a distal end of the outer sleeve 106. The connector housing 108 is configured to releasably secure an end effector, e.g., the end effector 30 (FIG. 1), to the adapter assembly 100. A drive assembly 110 (FIG. 16) including first and second drive assemblies 112, 114 extends through the outer sleeve 106 of the adapter assembly 100. For a detailed description of the structure and function of an exemplary drive assembly, please refer to the '254 patent.

The adapter assembly 100 further includes a trocar assembly 120, and a retaining mechanism (not shown) for releasably securing the trocar assembly 120 within the distal portion of the adapter assembly 100. The trocar assembly 120 and the retaining mechanism will only be described to the extent necessary to describe the aspects of the disclosure. For a detailed description of the structure and function of an exemplary trocar assembly, please refer to the '254 patent. For a detailed description of the structure and function of exemplary retaining mechanism, please refer to U.S. Pat. Nos. 10,111,684, 10,524,797, and 10,702,302.

The trocar assembly 120 of the adapter assembly 100 includes a trocar housing 122, a trocar member 124 slidably disposed within the trocar housing 122, and a drive screw 126 operably received within the trocar member 124 for axially moving the trocar member 124 relative to the trocar housing 122. The trocar housing 122 may include one or more features for releasably connecting the trocar assembly 120 within the distal portion 104 of the adapter assembly 100. For example, and as shown, the trocar housing 122 defines first and second locking openings 123 (only one shown) for receiving respective first and second retention members (not shown) of the retaining mechanism (not shown) of the adapter assembly 100.

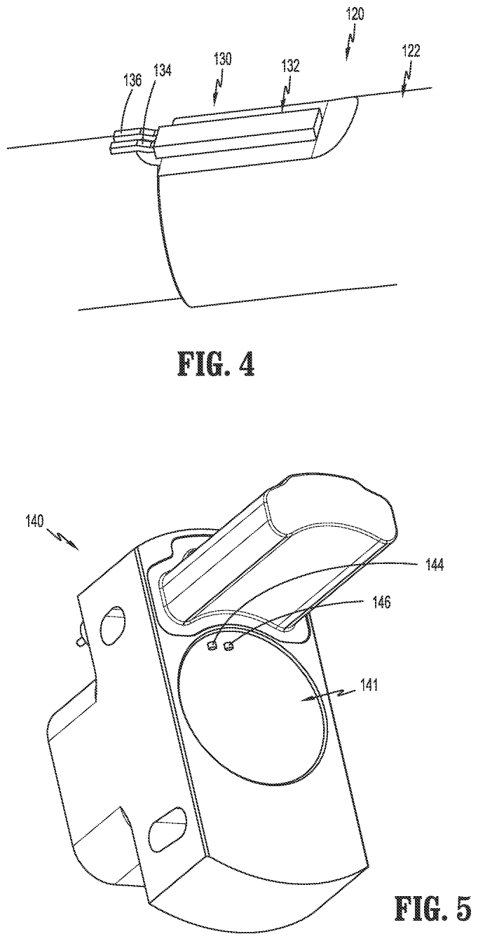

FIG. 4 illustrates a chip assembly 130 supported on the trocar housing 122 of the trocar assembly 120. The chip assembly 130 includes a chip 132 and a first and second contact members 134, 136 extending from the chip 132. In certain aspects of the disclosure, the chip 132 includes an erasable programmable read only memory (“EPROM”) chip. In other aspects of the disclosure, the chip 132 is a 1-wire chip. The chip 132 may be a read/write memory chip, such as read/write RAM, such that data may be written onto the chip 132.

The chip 132 may store data regarding the specifications of the trocar assembly 120. For example, the data may include the dimensions of the trocar assembly 120, e.g., the thickness of the trocar member 124 (FIG. 4), the length of the trocar member 124, the pitch of the thread of the drive screw 126. The chip 132 may include software for authenticating the trocar assembly 120 with the handle assembly 20 (FIG. 1). In this manner, the handle assembly, e.g., the handle assembly 20 (FIG. 1), may be configured to lock-out from use or disable when the trocar assembly 120 is not compatible with the handle assembly 20. The chip 132 may include a software for tracking the number of times the trocar assembly 120 is used, and a safety feature for preventing the trocar assembly 120 from being used beyond its recommended life.

The information provided to the handle assembly 20 by the chip 132 of the chip assembly 130 may be projected to the user audibly, e.g., beeps, alarms, tones, visually, e.g., display (not shown), L.E.D.s, and/or hepatically, e.g., vibrating handle. It is envisioned that the alerts may be programed to the clinicians specifications.

FIG. 5 illustrates a strain gauge assembly 140 for measuring strain experienced by the trocar assembly 120 during a surgical stapling procedure. It is envisioned that any known or commercially available strain gauge assemblies may be modified for use with the aspects of the disclosure. The strain gauge assembly 140 includes a housing 142. The housing 142 defines a longitudinal passage 141 for receiving the trocar assembly 120. First and second contact members 144, 146 extend from the housing 142 into the longitudinal passage 141 of the housing 142. The first and second contact members 144, 146 of the strain gauge assembly 140 are positioned to engage the first and second contact members 134, 136, respectively, of the trocar assembly 120 when the trocar assembly 120 is received within the distal portion 104 of the adapter assembly 100.

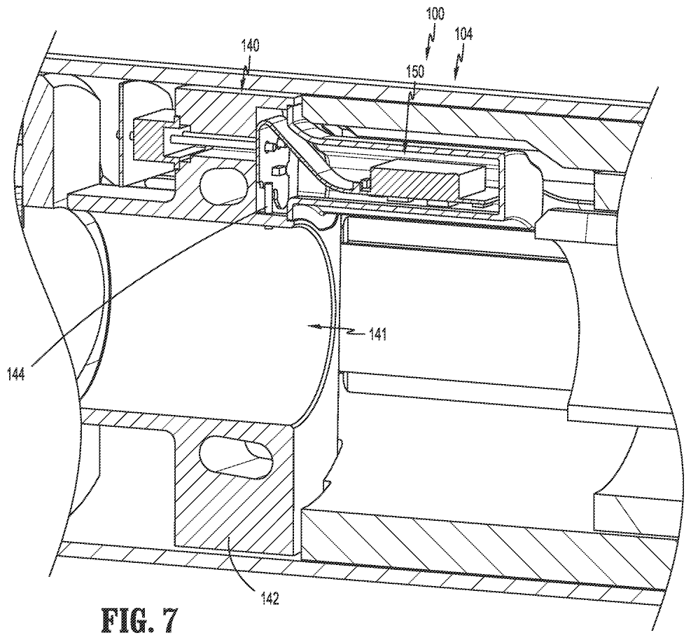

The first and second contact members 144 146 of the strain gauge assembly 140 are in electrical communication with a circuit board 150. The circuit board 150 (FIG. 7) may be a part of the circuitry for measuring strain by the strain gauge assembly 140, or the circuit board 150 may be a stand-alone component. The circuit board 150 may include a processor, relay, or other components for reading and/or writing to the chip 132 of the chip assembly 130 supported by trocar assembly 120. The circuit board 150 is in electrical communication with the handle assembly 20 (FIG. 1). In some aspects of the disclosure, the communication between the chip assembly 130 and the handle assembly 20 is wireless, e.g., Bluetooth, Wi-Fi, radio waves.

Although shown as being part of the strain gauge assembly 140, it is envisioned that the circuit board assembly 150, and the corresponding first and second contact members 144, 146 of the strain gauge assembly 140, may be supported elsewhere within the distal portion 104 of the adapter assembly 100 for engagement with the chip assembly 130 of the trocar assembly 120 when the trocar assembly 120 is received within the distal portion 104 of the adapter assembly 100. In other aspects of this disclosure, the chip 132 of the chip assembly 130 is communicates with the handle assembly 20 (FIG. 1) directly, wirelessly or otherwise.

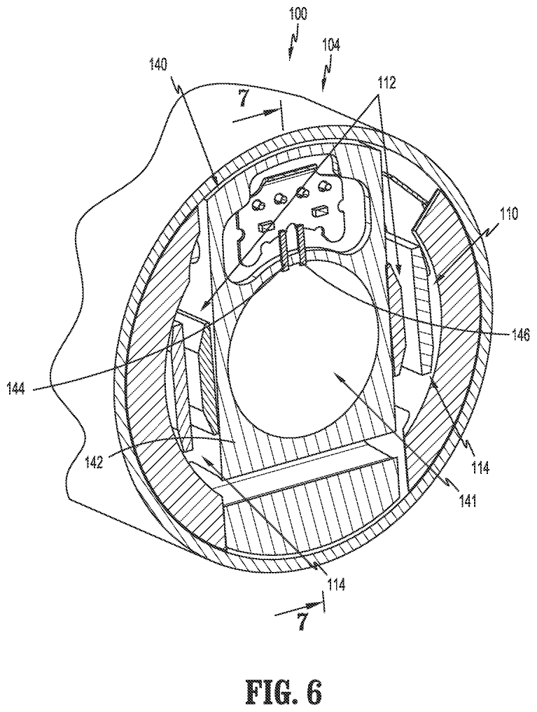

FIGS. 6 and 7 illustrate the adapter assembly 100 prior to receipt of the trocar assembly 120 within the distal portion 104 of the adapter assembly 100. As shown, the first and second contact members 144, 146 of the strain gauge assembly 140 extend from the housing 140 into the longitudinal opening 141 of the housing 142.

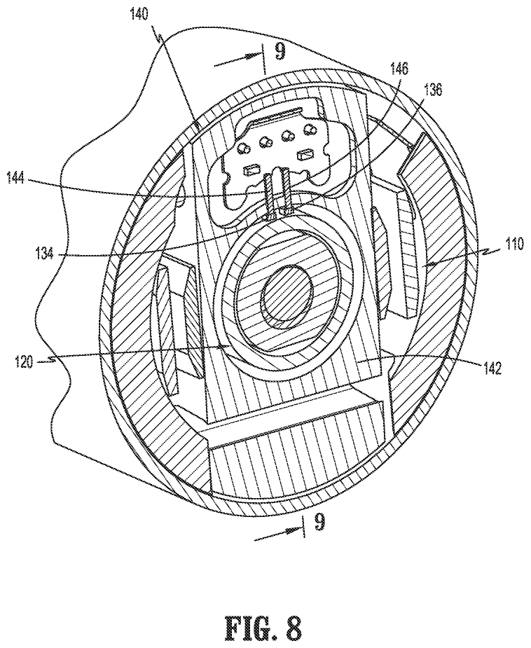

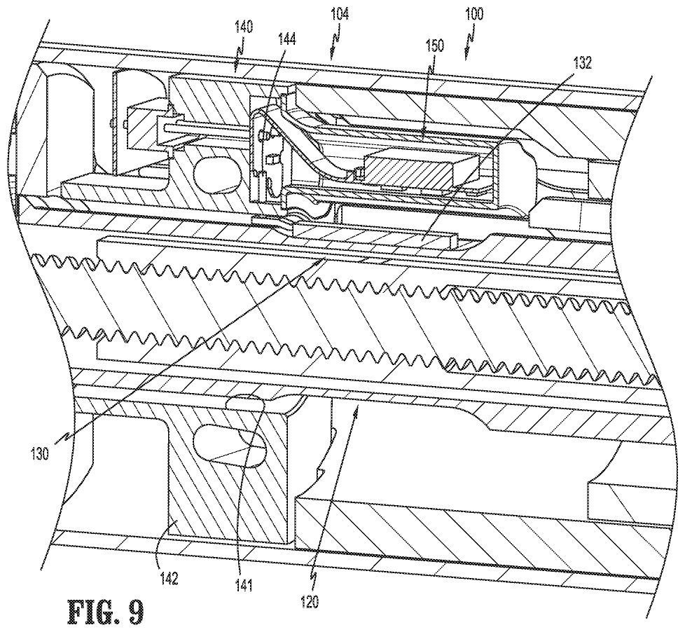

FIGS. 8 and 9 illustrate the adapter assembly 100 and the trocar assembly 120 received within the distal portion 104 of the adapter assembly 100. When the trocar assembly 120 is received within the distal portion 104 of the adapter assembly 100, the first and second contact members 144, 146 of the strain gauge assembly 140 engage the respective first and second contact members 134, 136 of the chip assembly 130 of the trocar assembly 120.

Upon receipt of the trocar assembly 120 within the distal portion 104 of the adapter assembly 100 and/or activation of the handle assembly 20 (FIG. 1), the information stored on the chip 132 of the chip assembly 130 is delivered to the handle assembly 20. Depending on the programming of the handle assembly 20, the user may receive any number of alerts and/or notifications regarding the configuration and/or condition of the trocar assembly 120.

Persons skilled in the art will understand that the devices and methods specifically described herein and illustrated in the accompanying drawings are non-limiting exemplary aspects of the disclosure. It is envisioned that the elements and features illustrated or described in connection with one exemplary aspect may be combined with the elements and features of another without departing from the scope of the disclosure. As well, one skilled in the art will appreciate further features and advantages of the disclosure based on the above-described aspects. Accordingly, the disclosure is not to be limited by what has been particularly shown and described.

What is claimed is:

1. An adapter assembly for connecting a loading unit to a handle assembly, the adapter assembly comprising:
an elongate body having a proximal portion and a distal portion, the proximal portion being configured for operable engagement with an actuation assembly and the distal portion defining a longitudinal axis; and
a trocar assembly releasably receivable within the distal portion of the elongate body, the trocar assembly including a chip for storing data.

2. The adapter assembly of claim 1, further including a housing supporting a circuit board, wherein the chip electrically communicates with the circuit board when the trocar assembly is received within the distal portion of the elongate body.

3. The adapter assembly of claim 2, wherein the housing is a component of a strain gauge assembly.

4. The adapter assembly of claim 1, wherein the trocar assembly includes a trocar housing, a trocar member, and a drive screw, and rotation of the drive screw in a first direction causes advancement of the trocar member relative to the trocar housing and rotation of the drive screw in a second direction causes retraction of the trocar member relative to the trocar housing.

5. The adapter assembly of claim 4, wherein the data includes a length of the trocar member.

6. The adapter assembly of claim 4, wherein the drive screw includes a thread having a pitch, wherein the data includes the pitch of the drive screw.

7. The adapter assembly of claim 4, wherein the data includes a diameter of the trocar member.

8. The adapter assembly of claim 1, wherein the data includes a number of times the trocar assembly has been used.

9. The adapter assembly of claim 1, wherein the trocar assembly includes first and second contact members and the elongate body includes first and second contact members, the first and second contact members of the trocar assembly being configured to engage with the respective first and second contact members of the elongate body when the trocar assembly is received within the distal portion of the elongate body.

10. The adapter assembly of claim 1, further including a circuit board assembly in electric communication with the chip when the trocar assembly is received within the distal portion of the adapter assembly.

11. A surgical stapling instrument, comprising:
an actuation assembly; and
an adapter assembly for connecting a loading unit to a handle assembly, the adapter assembly including,
an elongate body having a proximal portion and a distal portion, the proximal portion being configured for operable engagement with an actuation assembly and the distal portion defining a longitudinal axis; and
a trocar assembly releasably receivable within the distal portion of the elongate body, the trocar assembly including a chip for storing data.

12. The surgical stapling instrument of claim 11, further including a housing supporting a circuit board, wherein the chip electrically communicates with the circuit board when the trocar assembly is received within the distal portion of the elongate body.

13. The surgical stapling instrument of claim 12, wherein the housing is a component of a strain gauge assembly.

14. The surgical stapling instrument of claim 11, wherein the trocar assembly includes a trocar housing, a trocar member, and a drive screw, and rotation of the drive screw in a first direction causes advancement of the trocar member relative to the trocar housing and rotation of the drive screw in a second direction causes retraction of the trocar member relative to the trocar housing.

15. The surgical stapling instrument of claim 14, wherein the data includes a length of the trocar member.

16. The surgical stapling instrument of claim 14, wherein the drive screw includes a thread having a pitch, wherein the data includes the pitch of the drive screw.

17. The surgical stapling instrument of claim 14, wherein the data includes a diameter of the trocar member.

18. The surgical stapling instrument of claim 11, wherein the data includes a number of times the trocar assembly has been used.

19. The surgical stapling instrument of claim 11, wherein the trocar assembly includes first and second contact members and the elongate body includes first and second contact members, the first and second contact members of the trocar assembly being configured to engage with the respective first and second contact members of the elongate body when the trocar assembly is received within the distal portion of the elongate body.

20. The surgical stapling instrument of claim 11, further including a circuit board assembly in electric communication with the chip when the trocar assembly is received within the distal portion of the adapter assembly.
